(19) United States
(12) Patent Application Publication
(10) Pub. No.: US 2006/0148833 A1
(43) Pub. Date: Jul. 6, 2006

(54) Use of chymase inhibitors for the prevention and/or treatment of arterio-venous graft failure

(76) Inventor: Robert W Schroff, Edmonds, WA (US)

Correspondence Address: Brian R Morrill;Biomeasure Incorporated, 27 Maple Street, Milford, MA, 01757, US

(21) Appl. No.: 10/523,311

(22) PCT Filed: Jul. 29, 2003
(86) PCT No.: PCT/US03/23456

§ 371 Date: Aug. 31, 2005

Related U.S. Application Data
(60) Provisional application No. 60/399,538, filed on Jul. 30, 2002.

(51) Int. Cl. A61K 31/513; A61K 31/195

(52) U.S. Cl. 514/269; 514/563

(57) ABSTRACT

Disclosed is a method of treating A-V graft failure in a subject in need of such treatment, said method comprising administering to said subject an effective amount of an agent that inhibits the production, release or neo-intima generating effects of chymase, wherein said effective amount of said agent is an amount effective to treat said A-V graft failure.

### BACKGROUND OF THE INVENTION

Approximately 100,000 arterio-venous (A-V) vascular access procedures are performed each year in the US to provide access for the performance of renal dialysis (Stanley et al., J. Vasc. Surg. 1996, 23, 172-181). The most common material used for prosthetic dialysis access is polytetrafluoroethylene (PTFE or Gore-Tex), but approximately 60% of these grafts fail each year, usually due to stenosis at the venous end (Culp et al., Am. J. Kidney Dis. 1995, 26, 341-346; Churchill et al., J. Am. Soc. Nephrol. 1994, 4, 1809-1813; and Feldman et al., Kidney Int. 1993, 43, 1091-1096). Similar lesions develop in PTFE grafts placed into the arterial circulation, and again there is a tendency for the distal end of the graft to be more effected, but the rate of stenosis is not as great as in A-V grafts (Cantelmo et al., J. Cardiovasc. Surg. (Torino) 1989, 30, 910-915). Studies have shown that stenosis in A-V grafts is associated with the proliferation and build-up of smooth muscle cells (Kohler et al., J. Vasc. Surg. 1999, 30, 744-751 and Rekhter et al., Arterioscler. Thromb. 1993, 13, 609-617).

Percutaneous transluminal coronary angioplasty (PTCA) is an established therapy for obstructive coronary artery diseases. PTCA may be accomplished by balloon dilation, or more recently by the use of intracoronary stents. The long-term efficacy of PTCA has been limited by the occurrence of restenosis (Lin et al., Circulation 1989, 79, 1374-1387). Restenosis following PTCA, like the stenosis that occurs following A-V graft placement, is characterized by the proliferation and migration of smooth muscle cells and the subsequent development of a neointima (Lin et al., supra). Angiotensin II has been shown to play a role in neointimal development (Dzau et al., Hypertension. 1991, 18(suppl II), II-100-II-105). The involvement of angiotensin II in the pathophysiology of restenosis following PTCA was subsequently confirmed by the demonstration that both angiotensin converting enzyme (ACE) inhibitors and angiotensin II antagonists inhibit neointima development following balloon angioplasty (Powell et al., Science 1989, 245, 186-188 and Osterrieder et al., Hypertension 1991, 18 (suppl II), II-60-II-64). Based on these observations, ACE inhibitors were used in clinical trials to prevent restenosis following PTCA, but were found to be ineffective (MERCATOR Study Group, Circulation 1992, 86, 100-110 and Faxon, J. Am. Coll. Cardiol. 1995, 25,362-369).

The failure of ACE inhibitors in clinical trials, in contrast to their efficacy in animal models, lead to investigations into the source of this discrepancy. The source of the discrepancy seemed to be related to species differences in angiotensin II formation in humans versus rats and other species commonly used in animal models of restenosis. While ACE plays an important role in the production of angiotensin II from angiotensin I in rat vascular tissue, the primary route for generation of angiotensin II in humans, monkey and dog vascular tissue is chymase (Okunishi et al., J. Hypertens. 1984, 2, 277-284; Okunishi et al., Biochem. Biophys. Res. Commun. 1987, 149, 1186-1192; Okunishi et al., Jpn. J. Pharmacol. 1993, 62, 207-210; Shiota et al., FEBS Lett. 1993, 323, 239-242; and Takai et al., FEBS Lett. 1997, 421, 86-90). Chymase from these species cleaves angiotensin I to produce angiotensin II, while chymase from rats degrades angiotensin I to inactive fragments (Le Trong et al., Proc. Natl. Acad. Sci. USA 1987, 84, 364-3679). Balloon injury of dog carotid arteries results in significant activation of vascular tissue chymase levels, but not ACE levels (Shiota et al., supra). While an ACE inhibitor has little inhibitory effect on the development of neointima following balloon injury in the dog carotid, significant inhibition of neointima development has been reported with the use of an angiotensin II receptor antagonist (Okunishi et al., J. Hypertens. 1994, 12(suppl 3), S132).

Chymase is produced primarily in connective tissue mast cells, and secreted into the interstitium. Some anti-allergenic drugs are capable of stabilizing mast cells, and thus inhibiting the release of chymase by mast cells. Tranilast, (N-(3,4-dimethoxycinnamoyl) anthranilic acid; available from A.G. Scientific, San Diego, Calif.) is an anti-allergy drug that stabilizes mast cells and mast cell degranulation (Okunishi et al., Jpn. J. Pharmacol. 1993, 62, 207-210 and Shiota et al., supra). Tranilast suppresses neointima formation in balloon-injured dog coronary arteries by suppression of vascular chymase levels (Okunishi et al., Jpn. J. Pharmacol. 1993, 62, 207-210; Shiota et al., supra; Takai et al., supra; and Le Trong et al., supra). Tranilast was subsequently shown upon oral administration over three months in clinical trials to markedly reduce the rate of restenosis following PTCA (Takai et al., supra).

In contrast to Tranilast, which inhibits chymase release by mast cells, NK3201, (2-(5-formylamino-6-oxo-3-phenyl-1,6,-dihydropyrmidine-1-yl)-N-{2,3-dioxo-6-(2-pyridyloxy)-1-phenylmethyl}hexyl acetamide) is a direct inhibitor of chymase activity (Takai et al., Circulation 2001, abstract number 1135; and U.S. Pat. No. 6,271,238). Similar to Tranilast, NK3201 has been shown to inhibit intimal hyperplasia in the dog carotid artery balloon injury model (Takai et al., Circulation 2001, abstract number 1135). In addition, NK3201 has demonstrated the ability to inhibit vascular proliferation and subsequent neointima formation in a dog model of vein graft injury (Takai et al., Life Sci. 2001, 69, 1725-1732). This model consists of bypass grafting of the carotid artery with a piece of the ipsilateral jugular vein. When the vein tissue is placed into the artery environment, the result is proliferation and neointima formation similar to that observed in the carotid artery balloon injury model.

### SUMMARY OF THE INVENTION

The present invention relates to the use of agents that inhibit the production, release or neo-intima generating effects of chymase for treating and/or inhibiting A-V graft failure. Accordingly, in a first aspect, the invention features a method of treating A-V graft failure in a subject, preferably a human, in need of such treatment that includes administering an effective amount of an agent that inhibits the production, release, or neo-intima generating effects of chymase to the subject, where the effective amount of the agent is that amount effective in treating the A-V graft failure. In one embodiment, the graft failure includes intimal hyperplasia, which can include the proliferation and migration of smooth muscle cells, such as can occur at the venous end of an A-V graft. Therefore, in another aspect, the invention features a method of treating intimal hyperplasia associated with an A-V graft by administering an agent that inhibits the production, release, or neo-intima generating effects of chymase. An example of an agent useful for any of the foregoing methods of the invention is N-(3,4-dimethoxycinnamoyl)anthranilic acid, or a pharmaceutically acceptable salt thereof. Other agents useful for any of the foregoing methods of the invention include angiotensin II receptor antagonists or chymase inhibitors, such as 2-(5-formylamino-6-oxo-3-phenyl-1,6,-dihydropyrmidine-1-yl)-N-{2,3-dioxo-6-(2-pyridyloxy)-1-phenylmethyl}hexyl acetamide.

### DETAILED DESCRIPTION

A-V graft failure displays intimal hyperplasia at the venous end of the graft that is similar in composition to that observed in animal models of arterial balloon injury and bypass grafting of a vein into an artery. Thus, compounds that show efficacy in these latter models would be useful in treating and/or inhibiting A-V graft failure. Importantly, although chymotrypsin type proteases have been considered to participate in some fashion in diseases such as asthma, allergy, inflammations, rheumatism, hypertension, heart failure, myocardial infarction, cardiac hypertrophy, vascular injuries accompanied by angiogenesis and atheroma, nephritis and renal insufficiency, the use of known inhibitors of neointimal development in PTCA restenosis models or vein graft stenosis models, e.g., chymase inhibitors, in the treatment and/or inhibition of A-V graft failure has not been previously suggested.

Chymase inhibitors are well known to those of skill in the art. A non-exclusive list of suitable chymase inhibitors would comprise, without limitation, the compounds described in the following references: U.S. Pat. Nos. 6,410,576; 6,372,744; 6,355,460; 6,271,238; 6,159,938; 6,080,738; 5,948,785; 5,814,631; 5,723,316; 5,691,335; 5,367,064; 5,266,465; 5,079,336; 5,723,316; and 6,271,238. Also well known are assays for determining chymase inhibitory activity (see, e.g., U.S. Pat. Nos. 6,410,576; 6,372,744; 6,355,460; 6,271,238; 5,723,316; 6,080,738; 5,948,785; 5,814,631; 5,723,316; and 5,691,335).

Preferred compounds for use in methods according to the invention would include, without limitation, angiotensin II receptor antagonists, mast cell stabilizing agents, and chymase inhibitors. Particularly preferred compounds for use in methods according to the invention would include TRANILAST and NK-3201, and pharmaceutically acceptable salts thereof. The synthesis of NK-3201 is detailed in U.S. Pat. No. 6,271,238 (see synthesis example No. 55 therein).

Compounds for use in methods according to the invention can be formulated and administered to a subject using the guidance provided herein along with techniques well known in the art. The preferred route of administration ensures that an effective amount of compound reaches the target. Guidelines for pharmaceutical administration in general are provided in, for example, Remington: The Science and Practice of Pharmacy 20^{th} Edition, Ed. Gennaro, Lippincott, Williams & Wilkins Publishing, 2000, which is hereby incorporated by reference herein.

Pharmaceutical compositions for use in the method of the invention may be formulated such that the pharmaceutically active compound is used alone or mixed with excipients or carriers and administered orally or parenterally such as by an injection, inhalant, tablets, granules, subtle granules, powder, capsules, suppositories, instillations, paste agents, ointments, sprays etc. As excipients or carriers, pharmaceutically acceptable additives are selected and the type and composition are determined according to the administration route and administration method. For example, in the case of an injection, sodium chloride or saccharides such as glucose, mannitol etc. is generally preferable. In the case of oral preparations, starch, lactose, crystalline cellulose, magnesium stearate etc. are preferable.

The content of the pharmaceutically active compound in a pharmaceutical composition varies depending on the preparation, but is usually in the range of 0.1% to 100% by weight, preferably 1% to 98% by weight. For example, in the case of an injection, the active ingredient is contained in the range of usually 0.1% to 30% by weight, preferably 1% to 10% by weight. In the case of an oral preparation, the pharmaceutically active compound is used with additives in the form of tablets, capsules, powder, granules, liquid, dry syrup etc. The capsules, tablets, granules and powder contain generally 5% to 100% by weight of the pharmaceutically active compound, preferably 25% to 98% by weight.

In general, an effective dosage of active ingredient may be varied. However, it is necessary that the amount of the active ingredient be such that a suitable dosage form is obtained. The selected dosage depends upon the desired therapeutic effect, on the route of administration, and on the duration of the treatment, all of which are within the realm of knowledge of one of ordinary skill in the art. Generally, dosage levels of between 0.0001 mg/kg to 100 mg/kg of body weight daily are administered to humans or other animals, e.g., mammals. A preferred dosage range is 0.01 mg/kg to 100.0 mg/kg of body weight daily, more preferably 1.0 mg/kg to 10.0 mg/kg of body weight daily, which can be administered as a single dose or divided into multiple doses, or provided for continuous administration.

Further, an effective dosage of active ingredient may be administered in a sustained release composition such as those described in the following patents: U.S. Pat. No. 5,672,659 teaches sustained release compositions comprising a bioactive agent and a polyester; U.S. Pat. No. 5,595,760 teaches sustained release compositions comprising a bioactive agent in a gelable form; U.S. application Ser. No. 08/929,363, filed Sep. 9, 1997, teaches polymeric sustained release compositions comprising a bioactive agent and chitosan; U.S. application Ser. No. 08/740,778, filed Nov. 1, 1996, teaches sustained release compositions comprising a bioactive agent and cyclodextrin; and U.S. application Ser. No. 09/015,394, filed Jan. 29, 1998, teaches absorbable sustained release compositions of a bioactive agent. The teachings of the foregoing patents and applications are incorporated herein by reference.

Other features and advantages of the present invention will be apparent from the present description and also from the claims. It is believed that one skilled in the art can, based on the description herein, utilize the present invention to its fullest extent. The following specific embodiments are therefore to be construed as merely illustrative and not limitative of the remainder of the disclosure in any way whatsoever. All of the documents cited herein are hereby incorporated by reference in their entirety.

From the above description, one skilled in the art can easily ascertain the essential characteristics of the present invention, and without departing from the spirit and scope thereof, can make various changes and modifications of the invention to adapt it to various uses and conditions. Thus, other embodiments are also within the claims.

What is claimed is:

1. A method of treating A-V graft failure in a subject in need of such treatment, said method comprising administering an effective amount of an agent that inhibits the production, release, or neo-intima generating effects of chymase to said subject, wherein said effective amount of said agent is an amount effective to treat said A-V graft failure.

2. A method according to claim 1 wherein said A-V graft failure comprises intimal hyperplasia.

3. A method of treating intimal hyperplasia associated with an A-V graft, said method comprising administering an agent that inhibits the production, release or neo-intima generating effects of chymase.

4. A method according to claim 1, wherein said agent is N-(3,4-dimethoxycinnamoyl)anthranilic acid, or a pharmaceutically acceptable salt thereof.

5. A method according to claim 1, wherein said agent is an angiotensin II receptor antagonist.

6. A method according to claim 1, wherein said agent is a chymase inhibitor.

7. A method according to claim 6, wherein said chymase inhibitor is 2-(5-formylamino-6-oxo-3-phenyl-1,6,-dihydropyrmidine-1-yl)-N-{2,3-dioxo-6-(2-pyridyloxy)-1-phenylmethyl}hexyl acetamide, or a pharmaceutically acceptable salt thereof.

8. A method according to claim 1, wherein said subject is human.

9. A method according to claim 1, wherein said treatment comprises inhibition of intimal hyperplasia.

10. A method according to claim 9, wherein said intimal hyperplasia comprises the proliferation and migration of smooth muscle cells.

11. A method according to claim 9, wherein said intimal hyperplasia occurs at the venous end of said A-V graft.

12. A method according to any one of claims 8-11, wherein said agent is 2-(5-formylamino-6-oxo-3-phenyl-1,6,-dihydropyrmidine-1-yl)-N-{2,3-dioxo-6-(2-pyridyloxy)-1-phenylmethyl}hexyl acetamide, or a pharmaceutically acceptable salt thereof.
